(19) United States
(12) Patent Application Publication
(10) Pub. No.: US 2007/0009612 A1
(43) Pub. Date: Jan. 11, 2007

(54) Method for administering a composition to an animal

(76) Inventor: Robert R. Barefoot, Morristown, AZ (US)

Correspondence Address: Stephen T. Sullivan;Suite 1140, 1850 North Central Avenue, Phoenix, AZ, 85004, US

(21) Appl. No.: 11/016,688

(22) Filed: Dec. 16, 2004

(51) Int. Cl. A61K 31/59; A61K 31/525; A61K 31/51; A61K 31/4415; A61K 31/455; A61K 36/02; A61K 33/10

(52) U.S. Cl. 424/687; 514/251; 514/168; 514/276; 514/350; 514/355; 514/458; 514/725; 424/195.17

(57) ABSTRACT

Methods are provided for administering a composition to an animal. The compositions include coral calcium, and also may include magnesium, calcified seaweed, Aquamin®, and at least one vitamin selected from a group consisting of vitamin A, B series vitamins such as B3, B5, vitamin C, D, and E.

### BACKGROUND OF THE INVENTION

1. Field of the Invention

The present invention relates to compositions and methods for administering a composition to an animal, e.g., to supplement the diet of and/or provide nutrients to the animal.

2. Description of the Related Art

The dietary and nutritional needs of human beings have enjoyed increasing publicity and development in recent years. Attention to the dietary and nutritional well being of animals, however, has received far less attention. This is particularly true of vitamins and minerals. Many animals, and particularly those commonly kept as pets, have substantial vitamin and mineral requirements to maintain good health and longevity.

Among these is the need for calcium. Calcium is important and in some cases essential to good health for many animals. Calcium is involved in one way or another in a wide range of metabolic and other physiological processes, and has been associated with treatment or improvement with respect to a range of disease processes or ailments. For those who attempt to supplement their pet's or animal's calcium intake, such supplementation often takes the form of providing foods having substantial amounts of calcium. Another approach involves administering a dietary supplement that includes small amounts of calcium.

The former approach has enjoyed relatively limited success for a number of reasons. Relatively few owners or caretakers, for example, are knowledgeable of specific foods rich in calcium. Moreover, this approach can fail to provide adequate calcium bioavailability and absorption, for example, if a sufficient amount of vitamin D is not present. The latter approach of administering supplements typically improves bioavailability, for example, by including appropriate amounts of vitamin D. This supplementation approach also has been limited, however, for example, in that the form of the supplements often do not yield high absorption rates for absorption into the body of the animal. This is particularly true of calcium, which usually is presented in a form that is largely insoluble in water and does not break down well in the gut or gastrointestinal tract. These supplements also in many cases lack other important vitamins and minerals, or do not provide them in appropriately bioavailable or absorbable form.

Compositions and methods therefore are needed that will provide bioavailable and absorbable amounts of calcium, alone or together with other vitamins, minerals and/or nutrients.

Accordingly, an object of the present invention is to provide such compositions and methods, wherein calcium can be delivered to the animal, alone or in combination with other vitamins, minerals and/or nutrients, in a sufficiently bioavailable and absorbable form.

Additional objects and advantages of the invention will be set forth in the description which follows, and in part will be apparent from the description, or may be learned by practice of the invention. The objects and advantages of the invention may be realized and obtained by means of the instrumentalities and combinations pointed out in the appended claims.

### SUMMARY OF THE INVENTION

To achieve the foregoing objects, and in accordance with the purposes of the invention as embodied and broadly described in this document, a method is provided for administering a composition to an animal. The method comprises providing the composition to comprise coral calcium, and administering composition to the animal.

Preferably but optionally, the composition administered according to this method comprises magnesium, preferably in a ratio of the amount of calcium to magnesium of about 2 to 1. The composition according to this method also optionally but preferably comprises calcified seaweed. It may comprise Aquamin®. The composition according to this method also optionally but preferably comprises at least one vitamin selected from a group consisting of vitamin A, B3, B5, C, D, and E. A B series vitamin, preferably comprising a folate, also preferably is included. In accordance with a preferred implementation of this method, the composition is provided and administered as a biscuit.

In accordance with another aspect of the invention, a method is provided for administering a composition to an animal. The method comprises providing the composition to comprise a calcium component, a cesium chloride component and a vitamin D component, wherein the calcium component comprises coral calcium, and administering the composition to the animal.

Again, optionally but preferably the composition administered according to this method comprises magnesium, preferably in a ratio of the amount of calcium to magnesium of about 2 to 1. The composition according to this method also optionally but preferably comprises calcified seaweed, and may comprise Aquamin®. The composition according to this method also optionally but preferably comprises at least one vitamin, such as those identified herein above. The composition also may be provided and administered as a biscuit.

In accordance with another aspect of the invention, a composition is provided for administration to an animal. The composition comprises a calcium component preferably comprising coral calcium, a cesium chloride component, and a vitamin D component.

The composition preferably but optionally comprises magnesium, once again, preferably but optionally where the ratio of the amount of calcium to magnesium is about 2 to 1. The composition preferably comprises calcified seaweed, and may comprise Aquamin®. The composition also may and preferably does comprise at least one vitamin, such as those described herein above. As with the other compositions described herein, it may be administered as a biscuit or like form.

### DETAILED DESCRIPTION OF THE PREFERRED EMBODIMENTS AND METHODS

Reference will now be made in detail to the presently preferred embodiments and methods of the invention. It should be noted, however, that the invention in its broader aspects is not limited to the specific details, representative devices and methods, and illustrative examples shown and described in this section in connection with the preferred embodiments and methods. The invention according to its various aspects is particularly pointed out and distinctly claimed in the attached claims read in view of this specification, and appropriate equivalents.

TABLE 1
Example 1, Composition, and Preferred Dosage for Large
Animals, e.g., Horses.

| | | % | | % | | |
|---|---|---|---|---|---|---|
| Ingredient | Amount/Bottle | Comp. | Upper | Comp. | Lower | % Comp. |
| Coral calcium | 227 g | 47.0 | 400 g | 82.8 | 50 g | 10.4 |
| Calcium | 57 g | | 100 g | | 12 g | |
| Magnesium | 27 g | | 48 g | | 6 g | |
| Strontium | 364 mg | | 176 mg | | 80 mg | |
| Aquamin ® | 226 g | 46.8 | 400 g | 82.8 | 50 g | 10.4 |
| Calcium | 77 g | | 136 g | | 17 g | |
| Magnesium | 5 g | | 9 g | | 1 g | |
| Cesium Chloride | 690 mg | 0.14 | 2,000 mg | 0.41 | 100 mg | 0.02 |
| Vitamin-A | 1,200,000 IU | | 3,000,000 IU | | 500,000 IU | |
| Vitamin-B3 (niacin) | 4,500 mcg | 0.0009 | 10,000 mcg | 0.002 | 2,000 mcg | 0.0004 |
| Vitamin-B5 | 2,225 mg | 0.46 | 5,000 mg | 1.0 | 1,000 mg | 0.21 |
| (pantothenic) | | | | | | |
| Vitamin-C | 27,000 mg | 5.6 | 100,000 mg | 20.7 | 10,000 mg | 2.1 |
| Vitamin-D | 150,000 IU | | 400,000 IU | | 100,000 IU | |
| Vitamin-E | 15,000 IU | | 60,000 IU | | 5,000 IU | |
| Folate | 90,000 mcg | 0.02 | 200,000 mcg | 0.04 | 50,000 mcg | 0.01 |
| Total | 483 g | 100 | | | | |

In accordance with one aspect of the invention, a method is provided for administering a composition to an animal. The method comprises providing the composition to comprise coral calcium, and administering composition to the animal. In accordance with presently preferred embodiments of the invention and presently preferred implementations of this method, Table 1 presents compositions and ranges for use in administering to animals. This illustrative composition comprises ten component ingredients. The second column of the table, described as “Amount/Bottle,” identifies the amount of each ingredient used to make a 483 gram batch. For larger batches, the amounts may be increased proportionally or linearly.

The first component according to this aspect of the invention is coral calcium. The coral calcium used here preferably comprises, consists essentially of, and more preferably consists of high grade marine coral calcium. Preferred sources of such high grade coral calcium are commercial suppliers that obtain their coral calcium from Okinawa, Japan. Such sources of coral calcium typically include biologically active calcium and magnesium in a preferred ratio of about 2 to 1, respectively. For example, typically the coral calcium comprises 20-25 percent calcium and 10-12.5 percent magnesium. The coral calcium also includes about 60 trace nutrient metals and marine microbes. As shown in Table 1, the coral calcium in this example includes strontium as well. The amount of coral calcium present in the presently preferred compositions according to this aspect of the invention are in the range of 10.4 to 82.2 percent of the total mass of the composition. In this preferred but merely illustrative example, high grade marine coral calcium is present in amount of about 227 grams for the 483 gram batch. This equates to about 47.0 percent of the total mass of the composition. The high grade marine coral calcium in turn includes about 57 grams of calcium, 27 grams of magnesium, 364 milligrams of strontium, and other trace metals.

The second ingredient of the compositions identified in Table 1 is Aquamin®, which is a commercially available product. It preferably is present in an amount ranging from about 10.4 to about 82.8 percent of the total composition mass. In the preferred composition of Table 1, it is present in the amount of about 226 grams. Of this amount, about 77 grams are calcium Aquamin®, 5 grams are magnesium Aguamin®, and trace metals again are included.

The compositions of Table 1 also comprises cesium chloride. This component is present in an amount of about 0.02 to 0.41 percent. In this example, it is present in an amount of about 690 mg, which equates to about 0.14 percent of the total composition mass. Cesium chloride is reported in the medical and scientific literature as providing a cancer retarding effect.

Compositions according to this preferred method and composition includes at least one vitamin selected from a group consisting of vitamin A, B3, B5, C, D, and E. B series vitamins and folate preferably are included. The amounts of these vitamins are shown in Table 1 for the preferred compositions.

The compositions as described in Table 1 may be provided in a number of different forms. These include, for example, powders, liquids or liquid suspensions, in pill, tablet or capsule form, and the like. In the presently preferred implementation of the method, the composition is dehydrated into powder form and then pressed into biscuits. This may include excipients, for example, such as those known in the art. When administered as biscuits, the size, mass, and other characteristics of the biscuit will depend upon the animal, the application, etc. In the presently preferred embodiments and method implementations, the biscuits for large animals such as horses have a mass of about 5 grams each. The daily dosage minimum can range from about 2 to about 5 biscuits per day. For smaller animals such as dogs and cats, the biscuits preferably have a mass of about 1 gram, and may be administered in amounts of one biscuit per day to about 2 biscuits per day for a 10-pound animal, and about 5 biscuits per day for a 50-pound animal.

Preferred daily dosages for the compositions described in Table 1 are provided in Table 2.

TABLE 2
Daily Dosages for Compositions of Table 1

| Ingredient | Low | High | Preferred |
|---|---|---|---|
| Coral calcium | 5,000 mg | 10,000 mg | 7,575 mg |
| Calcium | | | 1,892 mg |
| Magnesium | | | 908 mg |
| Strontium | | | 12 mg |
| Aquamin | 5,000 mg | 10,000 mg | 7,523 mg |
| Calcium | | | 2,561 mg |
| Magnesium | | | 180 mg |
| Cesium Chloride | 10 mg | 40 mg | 23 mg |
| Vitamin-A | 10,000 IU | 100,000 IU | 40,000 IU |
| Vitamin-B3 (niacin) | 50 mcg | 400 mcg | 150 mcg |
| Vitamin-B5 (pantothenic) | 25 mg | 200 mg | 75 mg |
| Vitamin-C | 500 mg | 2,500 mg | 900 mg |
| Vitamin-D | 3,000 IU | 20,000 IU | 5,000 IU |
| Vitamin-E | 100 IU | 2,000 IU | 500 IU |
| Folate | 2,000 mcg | 5,000 mcg | 3,000 mcg |
| Total | 10.54 g | 22.75 g | 16.10 g |

As shown in Table 2, preferred daily dosage for compositions according to this aspect of the invention preferably are in a range of about 10.54 to 22.75 grams per day for large animals, e.g., horses, with the presently preferred daily dosage being about 16.10 grams. The preferred daily dosages for the presently preferred composition according to this aspect of the invention for larger animals is shown in the right-most column of Table 2. The amounts of the component ingredients also are shown. The daily dosages for smaller animals, such as dogs, cats, etc., would be about 20% of the large animal dosages. These dosages can be scaled depending, for example, on the mass of the animal.

In accordance with another aspect of the invention, a method is provided for administering a composition to an animal. The method comprises providing the composition to comprise a calcium component, a cesium chloride component and a vitamin D component, wherein the calcium component comprises coral calcium, and administering the composition to the animal.

Again, optionally but preferably the composition administered according to this method comprises magnesium, preferably in a ratio of the amount of calcium to magnesium of about 2 to 1. The composition according to this method also optionally but preferably comprises calcified seaweed, and may comprise Aquamin®. The composition according to this method also optionally but preferably comprises at least one vitamin, such as those identified herein above. The composition also may be provided and administered as a biscuit.

A presently preferred implementation of this method comprises administering one or more of the compositions described in Table 1 to the animal, preferably in the daily dosage ranges shown in Table 2. The composition may be provided and administered as described herein above.

In accordance with another aspect of the invention, a composition is provided for administration to an animal. The composition comprises a calcium component comprising coral calcium, a cesium chloride component, and a vitamin D component. The composition preferably but optionally comprises magnesium, once again, preferably but optionally where the ratio of the amount of calcium to magnesium is about 2 to 1. The composition preferably comprises calcified seaweed, and may comprise Aquamin®. The composition also may and preferably does comprise at least one vitamin, such as those described herein above. As with the other compositions described herein, it may be administered as a biscuit or like form.

Preferred compositions according to this aspect of the invention are described in Table 1 hereof Tables 3 and 4 provide additional illustrative examples of compositions according to these aspects of the invention. It is preferred that all ingredients as described herein are from natural sources.

Additional advantages and modifications will readily occur to those skilled in the art. Therefore, the invention in its broader aspects is not limited to the specific details, representative devices and methods, and illustrative examples shown and described. Accordingly, departures may be made from such details without departing from the spirit or scope of the general inventive concept as defined by the appended claims and their equivalents.

TABLE 3
Example 2, Composition, and Preferred Dosage for Large
Animals, e.g., Horses

| Ingredient | Amount/Bottle | Daily Intake |
|---|---|---|
| Coral calcium SMP | 110 g | 3,667 mg |
| Calcium | 28 g | 933 mg |
| Magnesium | 13 g | 440 mg |
| Strontium | 176 mg | 6 mg |
| Aquamin ® | 110 g | 3,667 mg |
| Calcium | 39 g | 1,300 mg |
| Magnesium | 3 g | 100 mg |
| Coral calcium CCP | 110 g | 3,667 mg |
| Calcium | 39 g | 1,300 mg |
| Magnesium | 2.2 g | 73 mg |
| Magnesium Citrate | 70 g | 2,333 mg |
| Magnesium | 5,586 mg | 186 mg |
| Magnesium oxide | 46 g | 1,533 mg |
| Magnesium | 28 g | 933 mg |
| Cesium Chloride | 690 mg | 23 mg |
| Vitamin-A | 1,200,000 IU | 40,000 IU |
| Vitamin-B3 (niacin) | 4,500 mcg | 150 mcg |
| Vitamin-B5 (pantothenic) | 2,225 mg | 74 mg |
| Vitamin-C | 27,000 mg | 900 mg |
| Vitamin-D | 150,000 IU | 5,000 IU |
| Vitamin-E | 15,000 IU | 500 IU |
| Folate | 90,000 mcg | 3,000 mcg |
| Total | 476 g | 15.87 g |

TABLE 4
Example 3, Composition, and Preferred Dosage for Large
Animals, e.g., Horses

| Ingredient | Amount/Bottle | Daily Intake |
|---|---|---|
| Coral calcium SMP | 110 g | 3,667 mg |
| Calcium | 27.5 g | 916 mg |
| Magnesium | 13 g | 440 mg |
| Strontium | 360 mg | 12 mg |
| Aquamin ® | 224 g | 7,461 mg |
| Calcium | 38.5 g | 1,283 mg |
| Magnesium | 3.4 g | 103 mg |
| Coral calcium CCP | 110 g | 3,667 mg |
| Calcium | 38.5 g | 1,283 mg |
| Magnesium | 2.2 g | 73 mg |
| Magnesium Citrate | 116 g | 3,867 mg |
| Magnesium | 5,586 mg | 186 mg |
| Magnesium oxide | 36 g | 1,200 mg |
| Magnesium | 30.8 g | 925 mg |
| Cesium Chloride | 690 mg | 23 mg |
| Vitamin-A | 1,200,000 IU | 40,000 IU |
| Vitamin-B3 (niacin) | 4,500 mcg | 150 mcg |
| Vitamin-B5 (pantothenic) | 2,250 mg | 74 mg |
| Vitamin-C | 27,000 mg | 900 mg |
| Vitamin-D | 150,000 IU | 5,000 IU |
| Vitamin-E | 15,000 IU | 500 IU |
| Folate | 90,000 mcg | 3,000 mcg |
| Total | 626 g | 20.86 g |

What is claimed is:

1. A method for administering a composition to an animal, the method comprising:
providing the composition to comprise coral calcium;
administering composition to the animal.

2. A method as recited in claim 1, wherein the composition providing comprises providing the composition to comprise magnesium.

3. A method as recited in claim 2, wherein the coral calcium comprises calcium, and the ratio of the amount of calcium to magnesium is about 2 to 1.

4. A method as recited in claim 2, wherein the composition providing comprises providing the composition to comprise calcified seaweed.

5. A method as recited in claim 1, wherein the composition providing comprises providing the composition to comprise Aquamin®.

6. A method as recited in claim 1, wherein the composition providing comprises providing the composition to comprise at least one vitamin selected from a group consisting of vitamin A, B3, B5, C, D, and E.

7. A method as recited in claim 1, wherein the composition providing comprises providing the composition to comprise vitamin A.

8. A method as recited in claim 1, wherein the composition providing comprises providing the composition to comprise at least one B series vitamin.

9. A method as recited in claim 1, wherein the composition providing comprises providing the composition to comprise a folate.

10. A method as recited in claim 1, wherein the composition providing comprises providing the composition to comprise vitamin C.

11. A method as recited in claim 1, wherein the composition providing comprises providing the composition to comprise vitamin D.

12. A method as recited in claim 1, wherein the composition providing comprises providing the composition to comprise vitamin E.

13. A method as recited in claim 1, wherein the composition providing comprises forming the composition into a biscuit.

14. A method for administering a composition to an animal, the method comprising:
providing the composition to comprise a calcium component, a cesium chloride component and a vitamin D component, wherein the calcium component comprises coral calcium; and
administering the composition to the animal.

15. A method as recited in claim 14, wherein the composition providing comprises providing the composition to comprise magnesium.

16. A method as recited in claim 15, wherein the coral calcium comprises calcium, and the ratio of the amount of calcium to magnesium is about 2 to 1.

17. A method as recited in claim 15, wherein the composition providing comprises providing the composition to comprise calcified seaweed.

18. A method as recited in claim 14, wherein the composition providing comprises providing the composition to comprise Aquamin®.

19. A method as recited in claim 14, wherein the composition providing comprises providing the composition to comprise at least one vitamin selected from a group consisting of vitamin A, B3, B5, C and E.

20. A method as recited in claim 14, wherein the composition providing comprises providing the composition to comprise vitamin A.

21. A method as recited in claim 14, wherein the composition providing comprises providing the composition to comprise at least one B series vitamin.

22. A method as recited in claim 14, wherein the composition providing comprises providing the composition to comprise a folate.

23. A method as recited in claim 14, wherein the composition providing comprises providing the composition to comprise vitamin C.

24. A method as recited in claim 14, wherein the composition providing comprises providing the composition to comprise vitamin E.

25. A method as recited in claim 14, wherein the composition providing comprises forming the composition into a biscuit.

26. A composition for administration to an animal, the composition comprising:
a calcium component comprising coral calcium;
a cesium chloride component; and
a vitamin D component.

27. A composition as recited in claim 26, further comprising magnesium.

28. A composition as recited in claim 26, wherein the coral calcium comprises calcium, and the ratio of the amount of calcium to magnesium is about 2 to 1.

29. A composition as recited in claim 26, wherein the composition comprises calcified seaweed.

30. A composition as recited in claim 26, wherein the composition comprises Aquamin®.

31. A composition as recited in claim 26, wherein the composition comprises at least one vitamin selected from a group consisting of vitamin A, B3, B5, C and E.

32. A composition as recited in claim 26, wherein the composition comprises vitamin A.

33. A composition as recited in claim 26, wherein the composition comprises at least one B series vitamin.

34. A composition as recited in claim 26, wherein the composition comprises a folate.

35. A composition as recited in claim 26, wherein the composition comprises vitamin C.

36. A composition as recited in claim 26, wherein the composition comprises vitamin E.

37. A composition as recited in claim 26, wherein the composition comprises a biscuit.
